(19) United States
(12) Patent Application Publication
(10) Pub. No.: US 2005/0267346 A1
(43) Pub. Date: Dec. 1, 2005

(54) Non-invasive blood component measurement system

(75) Inventors: Ralf T. Faber, Lexington, MA (US); Erik J. Schwendeman, Charlton, MA (US); Guangming Wang, Bedford, MA (US)

Correspondence Address: EDWARDS & ANGELL, LLP, P.O. BOX 55874, BOSTON, MA, 02205, US

(73) Assignee: 3Wave Optics, LLC

(21) Appl. No.: 11/048,005

(22) Filed: Jan. 31, 2005

Related U.S. Application Data
(60) Provisional application No. 60/540,663, filed on Jan. 30, 2004.

(51) Int. Cl. A61B 5/00

(52) U.S. Cl. 600/322; 600/310

(57) ABSTRACT

Non-invasive, optical apparatus and methods for the direct measurement of hemoglobin derivatives and other analyte concentration levels in blood using diffuse reflection and transmission spectroscopy in the wavelength region 400-1350 nm which includes the transparent tissue window from approximately 610 to 1311 nanometers and, using diffuse reflection spectroscopy, the mid-infrared region from 4.3-12 microns in wavelength. Large area light collection techniques are utilized to provide a much larger pulsate signal than can be obtain with current sensor technology. Sensors used in separate or simultaneous precision measurements of both diffuse reflection and transmission, either separately or simultaneously, from pulsate, blood-perfused tissue for the subsequent determination of the blood analytes concentrations such as arterial blood oxygen saturation (SaO₂), carboxyhemoglobin (COHb), oxyhemoglobin (OHb), deoxyhemoglobin (dOHb), methemoglobin (metHb), water (H2O), hematocrit (HCT), glucose, cholesterol and proteins such as albumin and other analytes components.

### CROSS-REFERENCE TO RELATED APPLICATIONS

This application claims the benefit of U.S. Provisional Patent Ser. No. 60/540,663 filed Jan. 30, 2004, the disclosure of which is incorporated herein by reference.

### FIELD OF THE INVENTION

This invention relates in general to the measurement and subsequent determination of solute concentrations. More specifically, it relates to a non-invasive, optical apparatus and method for the direct simultaneous measurement and monitoring of blood constituents.

### BACKGROUND OF THE INVENTION

While many medical procedures in hospitals are using non-invasive technology, the measurement and monitoring of blood constituents is still an invasive procedure which requires the drawing of blood.

Although the chemical blood analysis in hospitals and doctors practices is well established and precise, it requires multiple expensive devices to determine the various blood components.

These devices might be in different locations within the hospital, which will make it time consuming and expensive to get the full information. This adds time to diagnosis and treatment which is critical in emergency situations. It also requires practice, training, logistics and administrative support to make this cumbersome process work.

While oxygen saturation measurement is taken non-invasively already, most of the other blood components have to be determined by blood analysis using blood samples drawn from the patient.

Blood Oxygen Saturation, SaO2

Conventional transmission pulse oximetry is a standard of care for many patient populations. The pulse oximeter also has become a vital instrument in the care of infants and children with cardio pulmonary disease.

Recent advances in pulse oximetry technology have improved some aspects of pulse oximetry performance. However, monitoring challenges persist. The reliability, accuracy and clinical utility of pulse oximetry remain problematic. For instance, patient care providers of hospitals have noticed a high incidence of false alarms. False alarms on oxygen saturation monitors present a serious patient safety issue, since they cannot be distinguished from true alarms.

Carboxyhemoflobin, COHB

The fast and cheep quantification of the carbon monoxide level in blood is another critical step, that can provide valuable information. For instance, the immediate measurement of carboxyhemoglobin in people who have been exposed to heavy smoke, like firefighters, could save lives. However, the device needs to be portable and easy enough to use in ambulance vehicle or fire trucks.

This technology could be used in a fast screening device, allowing doctors the early detection and monitoring of lung cancer. As is well known, the carboxyhemoglobin in cigarette smokers can increase up to 15% of the total hemoglobin, while it is less than 3% in a normal healthy person.

Blood Glucose

Many approaches of non-invasive blood glucose measurement have been suggested over the years. Known apparatus and techniques operate on a wide variety of principles such as spectroscopy, refractometry, total internal reflection, polarimetry, etc. Any blood glucose measuring system, however, must address certain problems and achieve certain performance criteria. A practical blood glucose measurement system for patient use should be reliable and accurate, preferably at least to within 10 mg/dL.

Sickle Cells

Sickle cell disease is a blood condition seen most commonly in people of African ancestry. Patients with a high concentration of sickle cells may experience an undersupply of oxygen, which can cause severe difficulties. Basically, decreasing the amount of sickle hemoglobin and increase the amount of fetal or normal hemoglobin by a variety of means could treat the disease. Therefore, a simple measure of how much sickle hemoglobin a patient has, might be of use in newborns and others who are having symptoms of sickle cell disease.

U.S. Pat. Nos. 5,313,941, 5,666,956 and 6,445,938 disclose optical, non-invasive blood glucose measurement systems.

U.S. Pat. No. 5,313,941 discloses a non-invasive sensing device that can be used for blood glucose determinations. Long wavelength range infrared energy is passed through an appendage (finger) containing venous or capillary blood flow. The infrared energy is synchronized with the diastole and systole phase of the cardiac cycle. The measurements are made by monitoring strong and distinguishable infrared absorption of the desired blood analyte. Applicants are not aware of any working device results from such a device that were presented to the public, nor any product of this type introduced for public use.

U.S. Pat. No. 5,666,956 describes another non-invasive device that uses the natural thermal infrared emission from the tympanic membrane (ear drum) to detect blood glucose concentration in human body tissue. A portion of this thermal radiation is collected and analyzed using various mid-infrared filtering schemes to a detector with further electronic processing. Results are shown for testing on a non-diabetic individual. Such a device developed by Infratec, Inc. has been clinically tested and reported in 2002.

U.S. Pat. No. 6,445,938 discloses a “method for determining blood glucose levels from a single surface of the skin”. A device using this method is described in the patent which uses attenuated total reflection (ATR) mid-infrared spectroscopy to measure blood glucose in the outer skin of a fingertip. Prototype devices using this method have been developed by MedOptix, Inc.

Detection of carboxyhemoglobin and met-hemoglobin concentrations in blood is important during emergency situations such as carbon dioxide poisoning due to smoke inhalation, residential heating systems, automobile exhausts as well as drug overdose. They are usually measured from invasively drawn arterial blood samples that are measured in a specialized spectrometer known as a CO-oximeter.

U.S. Pat. Nos. 6,115,621, 6,397,093 B1 and 6,104,938 disclose optical, non-invasive oximeter measurement systems that attempt to address these issues.

U.S. Pat. No. 6,115,621 describes an oximeter sensor that uses an offset light emitter and detector. It increases the diffused light optical path length through the blood-perfused tissue by incorporating a reflective planer surface on each tissue exposed side of the sensor. Sensor designs are shown for application to the ear lobe and nose.

U.S. Pat. No. 6,397,093 B1 describes using a modified conventional, two wavelength pulse oximeter and sensor to measure carboxyhemoglobin non-invasively. Various predetermined calibration curves are used in the analysis.

U.S. Pat. No. 6,104,938 describes the apparatus and method to measure fractional oxygen saturation (OHb/total Hb) non-invasively. Four wavelengths in the red and near-infrared are used in the oximeter sensor design. Measurements can be made in either transmission or reflection.

### SUMMARY OF THE INVENTION

This invention relates in general to apparatus and methods used in precision measurements of diffuse reflection and transmission electromagnetic radiation, either separately or simultaneously, from pulsate, blood-perfused tissue for the subsequent determination of the blood analytes concentrations such as arterial blood oxygen saturation (SaO₂), carboxyhemoglobin (COHb), oxyhemoglobin (OHb), deoxyhemoglobin (dOHb), methemoglobin (metHb), water (H2O), hematocrit (HCT), glucose, cholesterol and proteins such as albumin. This diffusely reflected and transmitted light includes some scattered light, but it is predominantly reflected or transmitted.

More specifically, it relates to non-invasive, optical apparatus and methods for the direct measurement of hemoglobin derivatives and other analyte concentration levels in blood using a) both diffuse reflection and diffuse transmission spectroscopy in the approximate wavelength region 400-1350 nm—which includes the transparent “tissue window” from approximately 610 to 1311 nanometers; and b) using diffuse reflection spectrometry and operating in the mid-infrared region, from 4.3-12 microns in wavelength. Large area light collection techniques are utilized to provide a much larger pulsate signal than can be obtain with current sensor technology.

In one form of the invention useful in the measurement of blood analytes in the mid-infrared (MIR) wavelength region typically from 5 to 10 micron, the device has four principal components:

A first component is a tunable MIR light source of n≧2 specific, discrete spectral bands consisting of either a light source with peak blackbody wavelength between 9 and 11 microns passing through spectral filters or a spectrometer, MIR diodes, Lead-salt lasers, and Distributive Feed Back (DFB) or Multi-mode Quantum Cascade Lasers (QCL), composed of three or more lasers.

A second component is a sensor that utilizes lenses and reflective optics to collect diffuse reflected and scattered light from the tissue site, containing spectral (light intensity) information about the whole blood's current glucose, proteins, water and blood analyte concentrations.

A third component is an analyzer with algorithms for computing blood analyte concentrations. One algorithm is an iterative constituent sequenced algorithm for correlating diffuse collected light signals with a set of blood constituents. Each constituent is associated with one of the n spectral bands, successively. The other algorithm is a residual least squares curve fitting algorithm that fits collected diffuse light signals from blood pulsate tissue to a curve.

A fourth component is output electronics that displays the current concentration levels measured for blood analytes. This information may be stored electronically in random access memory (RAM) or other digital storage media for retrieval at a later time.

In another form of the present invention, an optical apparatus and methods for the direct measurement of hemoglobin derivatives and other analyte concentration levels in blood uses both diffuse reflection and diffuse transmission spectroscopy in the approximate wavelength region 400-1350 nm, which includes the transparent “tissue window” from approximately 610 to 1311 nanometers.

This form of the invention also has four principal components.

One component is a light emitter consisting of Quartz halogen, white light LED, discrete wavelength LEDs or diode lasers.

A second component is a pair of detectors with optics that collect the diffusely transmitted and reflected light from the blood-perfused tissues. The transmission detector is optimally located and facing the emitter so that it most efficiently collects the diffuse light from tissue (e.g. finger, earlobe, toe, or foot) placed between detector and emitter. The reflection detector is facing the illuminated tissue from the emitter and is located next to the emitter with an optimal separation. Both detectors may consist of silicon photodiodes and optics such as multimode fiber, lens, lenses, or optimized reflectors of parabolic or ellipsoidal shape. The output signals from each of the sensor's two detectors are proportional to light intensity. These signals are sent by multimode fibers or electrical cable to the analyzer for further analysis.

A third component is an analyzer which may consist of a personal computer and Digital Signal Processor (DSP) board or standard oximeter electronics. Computational analysis incorporates algorithms based on either an exactly determined or over-determined system of equations to calculate the total and ratio of concentrations of the blood analytes.

A fourth is an output electronics which may include display and audio-visual alarm electronics for “real time” results and digital storage using read-only memory (ROM for digital storage (results, trends, alarms, etc.)

These and other features and objects of the present invention will be more fully understood from the following detailed description of the invention, which should be read in light of the accompanying drawings.

### BRIEF DESCRIPTION OF THE DRAWINGS

FIG. 1 shows in schematic form one form of the apparatus for non-invasive analysis of blood components in the mid-infrared wavelength region;

FIG. 2a shows a schematic representation of a typical linear variable bandpass filter's physical configuration and spectral characteristics for use in the apparatus of FIG. 1;

FIG. 2b shows a schematic representation of a typical circular variable bandpass filter's physical configuration and spectral characteristics;

FIG. 2c shows a schematic representation of a typical discrete bandpass filter's physical configuration and spectral characteristics;

FIG. 3 shows in a schematic form various blood flow volume change due to cardiac cycle and body site clamping;

FIG. 4 shows a schematic of a diffuse reflection light collection system for use with an FT-IR Spectrometer as the light source in a mid-range non-invasive apparatus otherwise of the general type shown in FIG. 1;

FIG. 5 shows a flow chart for determining the blood analyte concentration illustrating one implementation of an iterative, constituent-sequenced algorithm for use with the apparatus of this invention;

FIG. 6 shows a flow chart for one form of a residual least squares algorithm for use with the apparatus of the invention to fit one component concentration using the collected diffuse light signals at a given wavelength or bandwidth associated with that one component;

FIG. 7 shows a Clarke Error grid analysis of measurement results for determining whole blood glucose concentration;

FIG. 8 shows a schematic of the invention apparatus for large area light collection of diffuse reflection and transmission from pulsate, blood-perfuse tissue;

FIG. 9 shows a graph of the absorbance versus wavelength spectra from 600 to 1100 nanometers of oxy (OHb) and deoxy (dHb) hemoglobin and liquid water;

FIG. 10 shows in schematic form an alternative embodiment of apparatus according to this invention for analysis of blood components in the visible, near infrared wavelength region using diffuse reflectance and transmission;

FIG. 11 shows a graph of the relative optical absorbance of four hemoglobin types versus wavelength in the visible and near infrared from 450 to 1000 nanometers;

FIG. 12 shows a graph of the relative optical absorbance of four hemoglobin types versus wavelength in the visible from 500 to 650 nanometers

### DESCRIPTION OF THE INVENTION

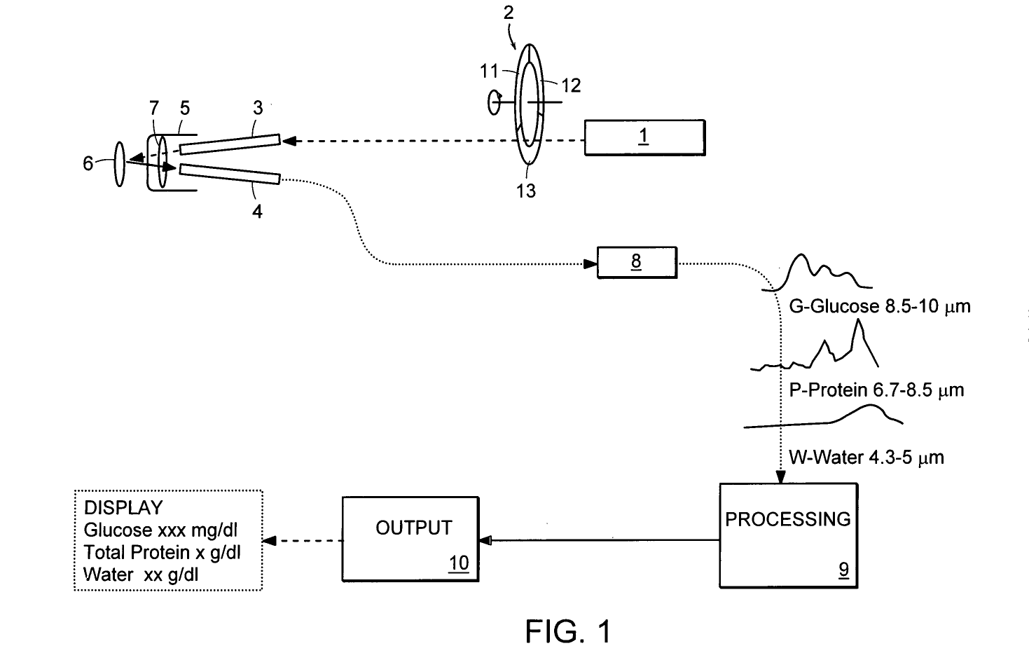

FIG. 1 shows in schematic form an apparatus particularly useful for an accurate, direct, non-invasive measurement of the blood glucose level. The invention is based on detecting and analyzing by diffuse reflection and optical spectroscopy the fundamental molecular vibrational modes of glucose, proteins and water in the mid-infrared (MIR) wavelength region from 5 to 10 micron.

MIR light from light source 1 such as ones available from Thermo-Oriel with spectral radiant emission peak blackbody wavelength between 9 and 11 microns passes through a rotating filter wheel 2 composed of spectral filters. Other technologies, such as MIR diodes, Lead-salt lasers, and Distributive Feed Back (DFB) or Multi-mode Quantum Cascade Lasers (QCL) may also be used as a tunable light source.

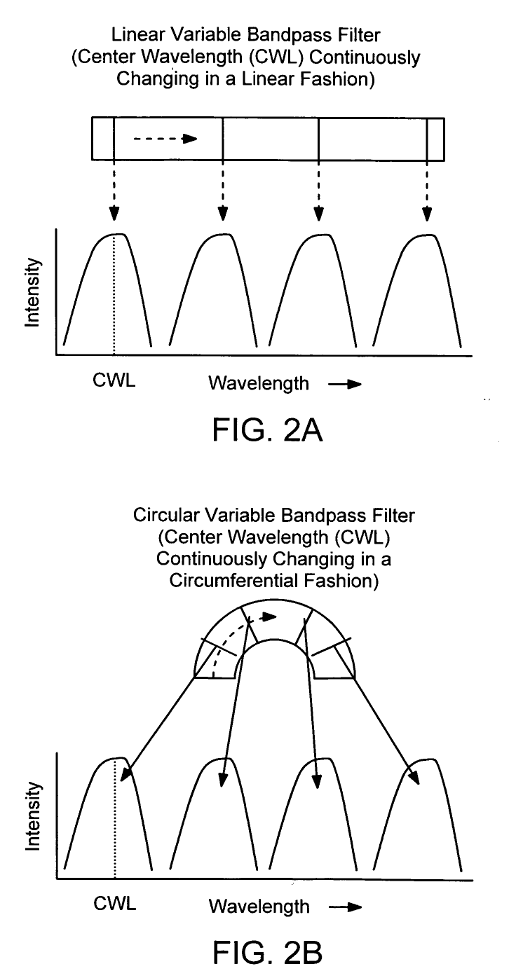

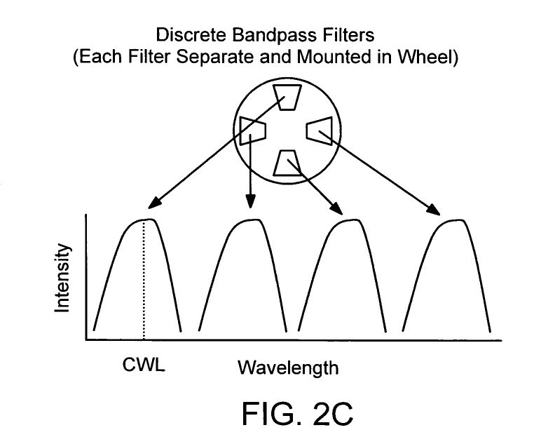

The filter wheel 2 is composed of three or more MIR optically transmitting filters. Typical variations of the wheel assembly are shown in FIGS. 2a, 2b and 2c. One filter 11 passes only the mid-IR light necessary for measuring glucose signal (8.5-10 micron). Another filter 12 passes only the mid-IR light necessary for measuring a protein signal (6.7-8.5 microns). The third filter 13 passes only the MIR light necessary to measure the water signal (4.3-5 μm). The filters 11, 12 and 13 are typically composed of multilayer thin films deposited onto an optically transmitting substrate. In addition, filters 11 and 12 are narrow bandpass circular variable (FIG. 2a), linearly variable (FIG. 2b) or discrete (FIG. 2c) filters with center wavelength from 6.7-10 micron while filter 13 is a broad bandpass filter centered from 4.3-5 micron. The rotation or movement of the filter wheel 2 is detected by a motor optical encoder (e.g. one from Encoder Products Co.) and synchronizing pulses with timing information (filter position at a given time) is sent to the processing unit 9. Other methods such as grating-dispersion based spectrometers from manufacturers such as Jobin-Yvon may be used to separate the glucose, protein and water MIR spectral regions.

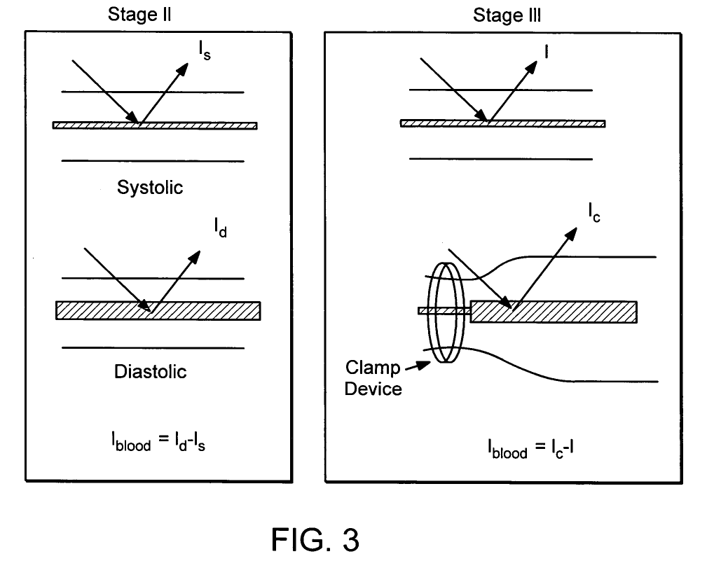

This filtered light is transmitted by a MIR optical light fiber/waveguide 3 such as one manufactured by such suppliers as CeramOptec or Amorphous Materials. It is focused by a MIR transmitting lens or lenses 7 through a plastic speculum 5 onto a body site 6 which contains capillary or venous blood to be analyzed. Blood volume at the site can be regulated by two suggested methods. One method is venous occlusion clamping, with inflation/deflation cuffs from D.E. Hokanson, Inc. or others, where venous blood flow from the site to the heart is stopped but arterial blood flow continues to the site from the heart. This stoppage increases blood pool volume with time the at the body site (FIG. 3). Measurements are made before and after clamping. Another method requires site measurements to be made in synchronization with the diastole and systole phases of the cardiac cycle (FIG. 3). A pulse oximeter with plethysmographic electronic output, for example one from Nellcor Puritan Bennett Inc., can be used for the trigger synchronization. Both methods allow spectral measurements to be made when blood volume at the site is a maximum and minimum. This will be used in the elimination of interfering effects of various intervening materials like tissue, melanin, collagen and fat.

The diffuse reflected and scattered light from the site, containing spectral (light intensity) information about the whole blood's current glucose, proteins and water concentration, is collected by the lens or lenses 7 and re-focused onto another MIR light optical fiber/waveguide 4.

The light is transmitted through an optical light fiber/waveguide 4 illuminating a high sensitivity mid-IR detector 8, typically composed of a Mercury Cadmium Telluride (HgCdTe, MCT) sensor element. MIR microbolometers, diode sensor element or arrays may also be used. The sensor may be cooled either thermoelectrically or with liquid nitrogen using a detector Dewar. In addition, the detector signal is further amplified with associated “pre-amp” electronics. A suitable detector of this type, with Dewar and pre-amp electronics, can be purchased from Judson Technologies.

The detector's amplified analog output from the mid-IR detector 8 is digitized by an analog-to-digital converter from such manufacturers as Analog Devices. This digital signal with its associated synchronized encoder timing information from the filter wheel 2 is sent to a Central Processing Unit/Digital Signal Processor, CPU/DSP 9 which performs further signal processing. An example of this device may consist of a personal computer and DSP PC board from Texas Instruments. Using the digitized detector/timing signal, the CPU/DSP 9 executes a computer code, written in such computer languages as Microsoft Visual Basic (VB). The encoder timing pulse from the filter wheel 2 is converted to a known MIR wavelength position. A two dimensional array is then calculated which consists of the wavelength and its corresponding intensity value from the detector 8 output. This array output forms three MIR spectrum (intensity versus wavelength) corresponding to measured blood glucose, protein and water.

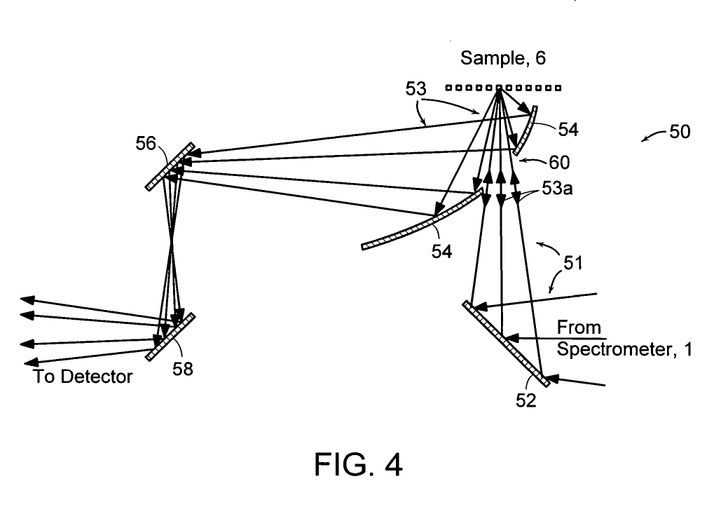

FIG. 4 shows apparatus 50 that can be used in the mid-IR measurement apparatus. It directs an interrogating beam 51 of radiation in the mid-IR range, produced by a spectrometer 1 (FIG. 1), to the tissue sample 6. It also collects the interrogating light diffusely reflected from the pulsating, blood-perfused tissue 6. A mirror 52 directs the interrogating beam from the spectrometer, through an opening 60, onto the sample 6. As shown, the angle of incidence of the light beam on the tissue is substantially normal. The light 53 scattered and diffusely reflected from the pulsating, blood-perfused tissue is intercepted by a reflector 54 that is 1) curved concavely with respect to the tissue, and 2) angled to direct the collected, diffusely reflected light 53 to a pair of planar mirrors 56, 58, which, in turn, direct this light onto a suitable light detector, such as the detector 8 in FIG. 1. The reflector 54 is preferably curved along an ellipsoidal path when viewed in cross-section as shown in FIG. 4.

The opening 60 within the reflector 54 both allows the interrogating beam 51 to pass through the reflector 54, and allows specular reflections from the sample to bypass detection and measurement by passing back through the opening 60, rather than being collected and directed to the detector 8. This specular reflection is indicated by arrow heads 53a.

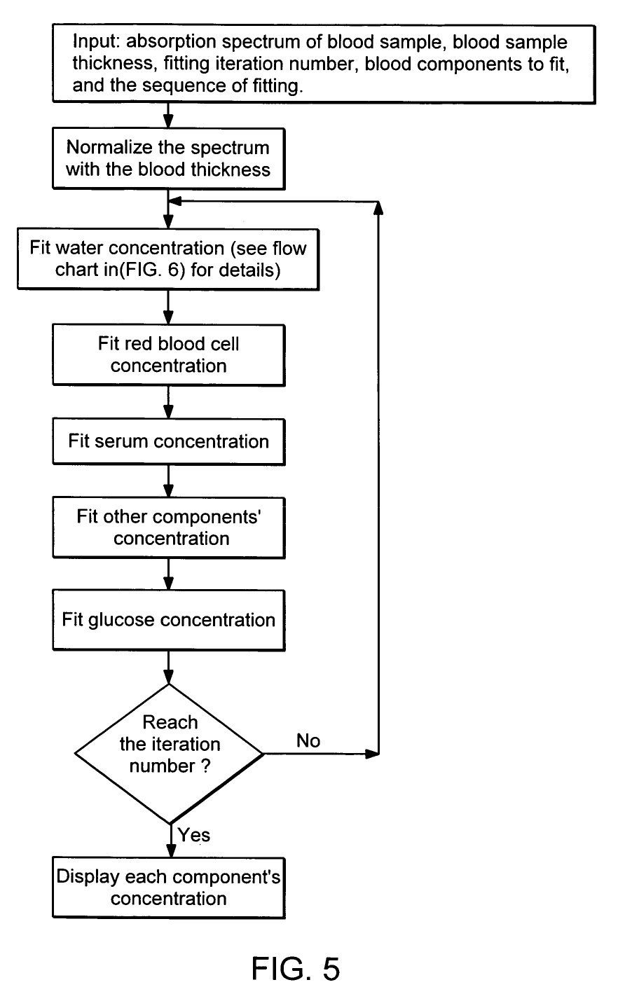

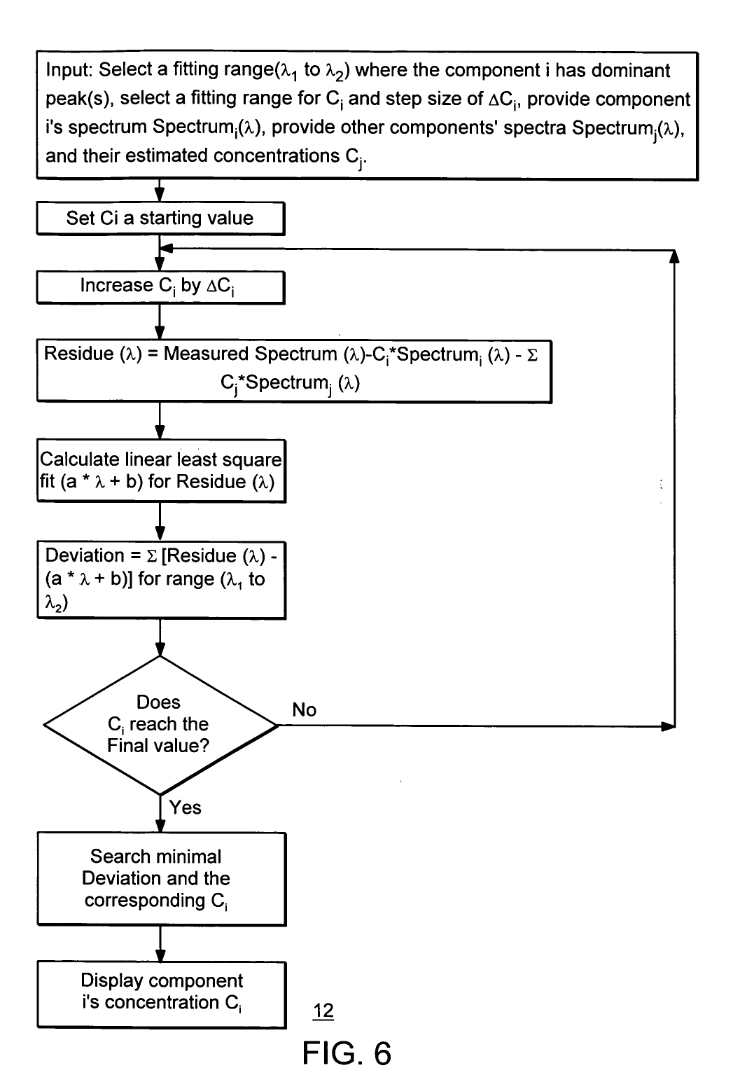

In operation, this apparatus eliminates interfering effects due to tissue, melanin, collagen and fat are eliminated by subtracting the spectrum at minimum blood volume from maximum blood volume at the body site. The resultant spectrum is the whole blood from the body site's capillaries or veins. Glucose, protein and water concentration in the whole blood are determined as follows. Analysis is performed by execution of additional computer code using flow chart shown in FIG. 5 written in such computer languages as Microsoft Visual Basic (VB). Each of n spectral regions (e.g. one each for glucose, protein and water) is compared to a corresponding glucose, protein and water calibration spectral data typically stored electronically in random access memory (ROM). The measured spectral intensities are multiplied by a constant and compared to their corresponding calibration spectrum intensity value until a least squares residual between the two spectra are minimized using the method shown in the flow chart of FIG. 6. This computed constant with the minimal residual is multiplied by the known calibration concentration and becomes the true concentration of the chemical in the whole blood of the body site. The method is reiterated many times for all components.

In the prior art, data at just a few wavelengths was used to calculate component concentrations in the blood. This practice is very difficult; among other reasons, because:
1. There are many components in the blood and their spectra overlap with each other. For example, the glucose peaks at 9-10 um region is overwhelmed by water base line and protein peaks.
2. Each component concentration is changing over time.
3. Some component concentrations are even lower than 0.1%.
4. There are noise, DC offset, and drift in the spectra due to instrument and sampling.

In the method depicted in FIG. 5, all spectra data over entire measurement range is used to provide the best fitting for all the components. This method converges fast to a global minimum in the fitting process.

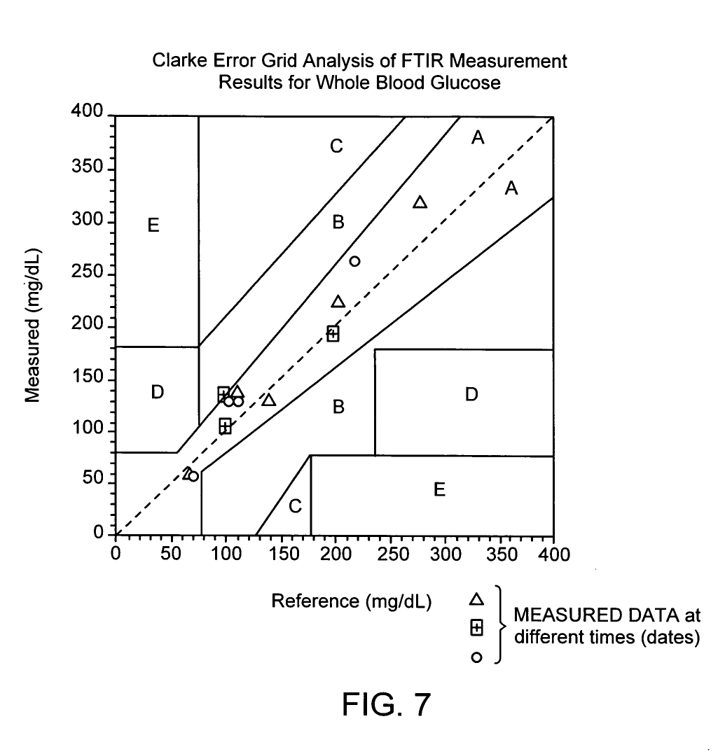

FIG. 7 is an example of actual in-vitro whole blood measurements using a Fourier Transform-Infrared (FT-IR) spectrometer and the analysis software plotted on a Clarke Error Grid. (From Clarke, W. L., et al., Diabetes Care, Vol. 10;5; 622-628 (1987), the disclosure of which is incorporated by reference.

In the Clark Error grid, zones A-E are defined as follows:
Zone A—Clinically accurate within ±20% of the Reference.
Zone B—Error greater than ±20%, but would lead to benign or no treatment.
Zone C—Errors would lead to unnecessary corrective treatment.
Zone D—Potentially dangerous failure to detect hypo- or hyperglycemia.
Zone E—Erroneous treatment of hypo- or hyperglycemia.

The output electronics 10 using e.g. liquid crystal (LCD) and or visible diode technologies displays the current concentration levels measured for blood glucose, protein and water. This information may be stored electronically in random access memory (RAM) or other digital storage media for retrieval at a later time.

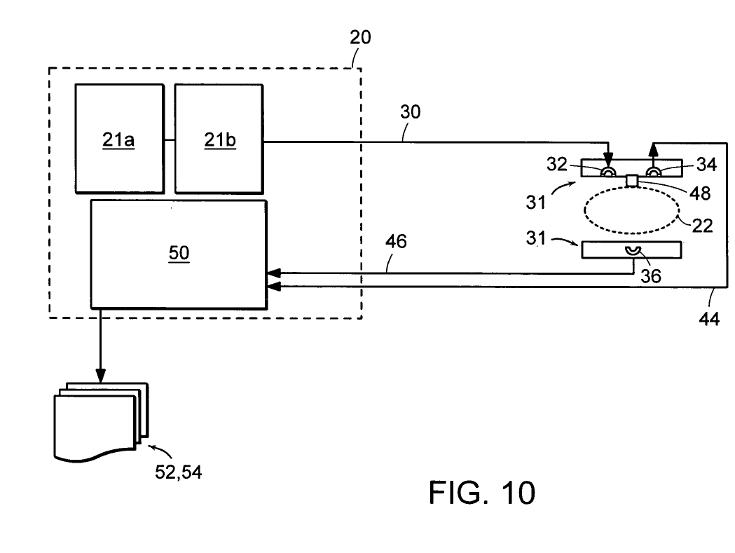

FIG. 10 shows in schematic form an apparatus 21 of the present invention particularly useful for an accurate, direct, non-invasive measurement of hemoglobin derivatives and other analyte concentrations in blood using interrogating radiation in the visible and near infrared, from approximately 400-1350 nanometers. The analyzer unit 1 may be portable or rack mounted.

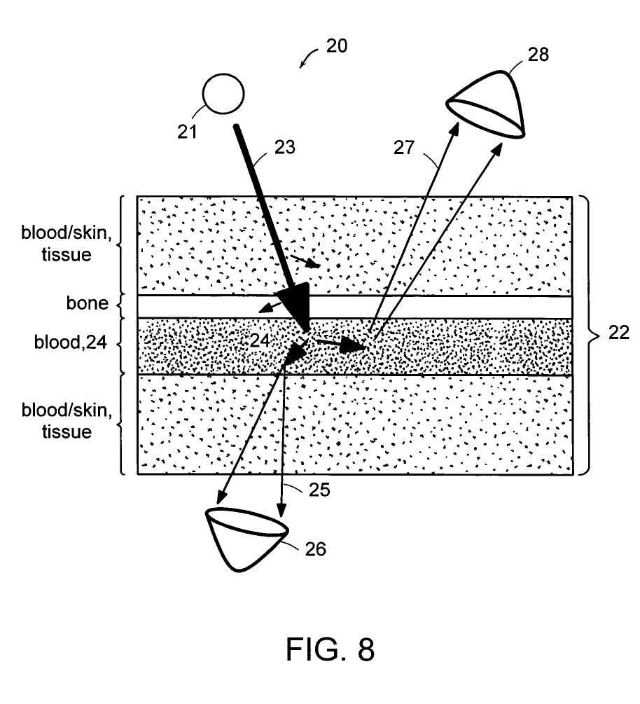

FIG. 8 shows this detection concept schematically. A multiple wavelength light source 21, consisting, for example, of a halogen bulb, LED, or diode laser illuminates a body part 22 such as a finger, toe or foot. The light passes through various layers which may include skin, blood (both venous and arterial pulsate), tissue, cartilage and bone. As the light passes through the body part it is absorbed and scattered. The scattered light from the arterial pulsate blood 24 is diffusely reflected 27 and transmitted 25 through the body part. Large area light collection detectors 26 and 28 capture this diffuse light for analysis.

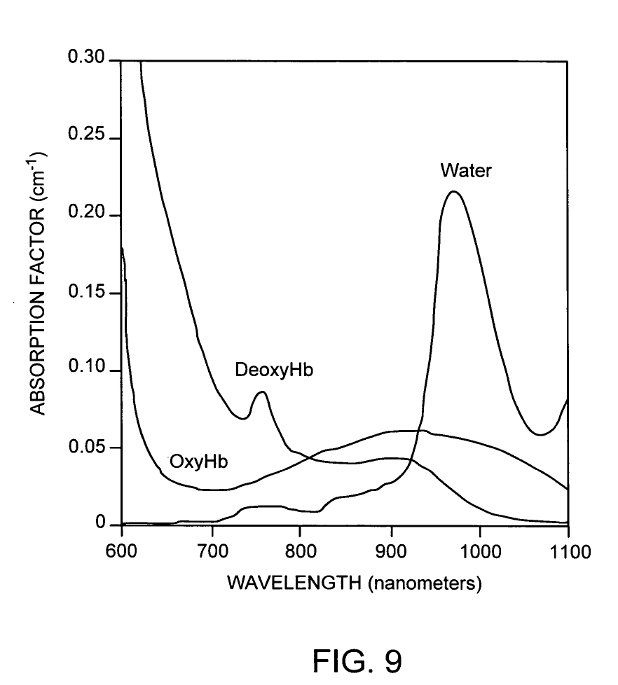

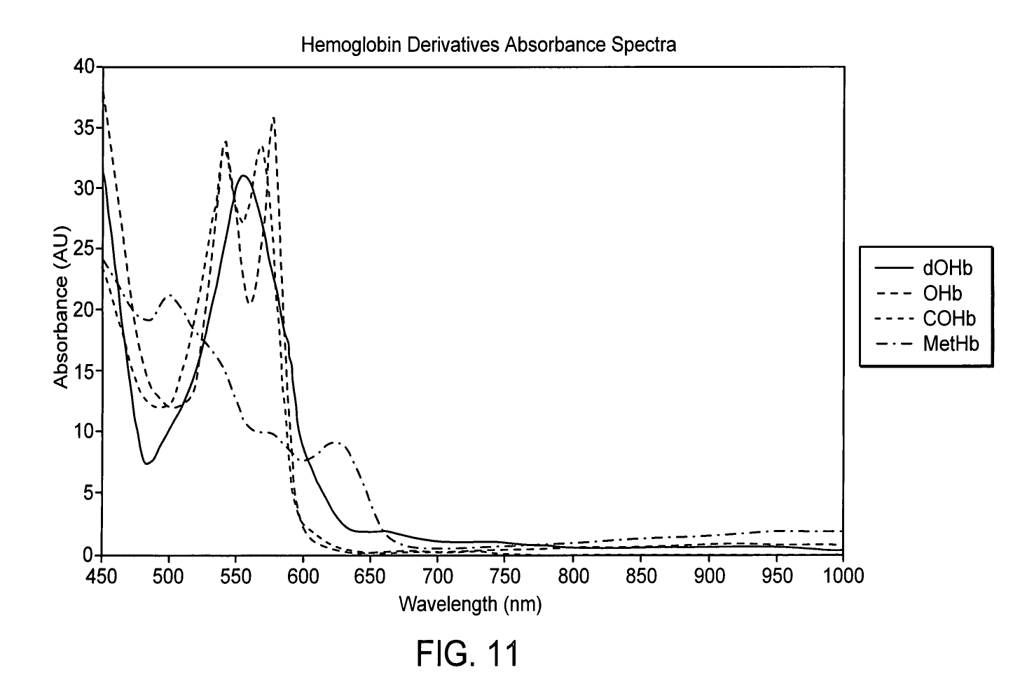

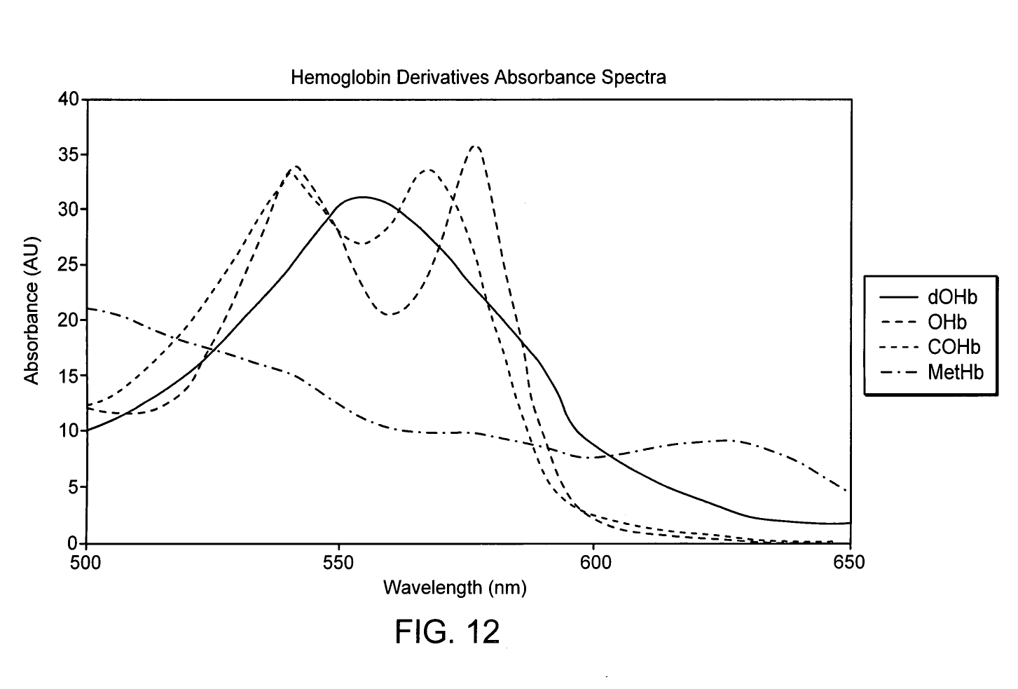

The apparatus 20 operates in the transparent “tissue window” from approximately 630 to 1350 nanometers in wavelength (see FIG. 11). Specific wavelengths are chosen which represent a particular analytes' unique light absorption properties (i.e. maximum absorbance) or regions where two analytes have identical absorbance (isosbestic point). Typical wavelengths used in the industry are 660, 800, 905 and 940 nm for transmission measurements of OHb and dOHb. Water has a unique absorption peak at 980 nanometer as shown in FIG. 9. Diffuse reflection measurements may include these wavelengths as well as the region of 530 to 619 nm shown in FIG. 12 where the hemoglobin derivatives optical absorbance is stronger and vary significantly from each other.

The light source 21 can be either of a broad band white light source 21a (Quartz halogen, white light LED), discrete wavelength LEDs or diode lasers with associated power supply. If a broadband white light source 21a or LEDs are used, then a spectrometer 21b with a diffraction grating or narrow bandpass filters is necessary to select specific, narrow wavelength regions from within the “tissue window”. A spectrometer 21b is not needed if wavelength specific LEDs or diode lasers are used. The light may be pulsed electronically or mechanically with a chopper to reduce the total amount of light radiation exposure to the tissue (typically less than 50 mW/cm2 continuous exposure). This light may be coupled by multimode optical fiber to the sensor input or emitter side.

A sensor unit 31 is comprised of an emitter 32 and two detectors 34, 36, each using optics incorporated into the sensor body to transmit (emitter) and collect the diffusely transmitted 25 and reflected light 27 from the blood-perfused tissues 22. The emitter optics may consist of multimode fibers, lens, lenses or optimized reflectors of parabolic or ellipsoidal shape. This optic is designed to maximize the collection of light from the source and to irradiate a much larger area of pulsate, arterial blood-perfused tissue than current technology oximeter sensors. The much larger area is usually at least twice, and typically is five times, as large as that of current oximetric sensors that are commercially available. This provides the detectors 34,36 with a stronger AC signal from this tissue as discussed below. Similarly, large core multimode fibers lens, lenses or optimized reflectors of parabolic or ellipsoidal shape collect the diffuse transmitted 25 and reflected light 27 emanating from the irradiated tissue 22 and couple it into multimode fibers 44 and 46, respectively. Direct light from the emitter is blocked from the diffuse reflector detector by an optical barrier 48. The solid angle collection area of the emitter and two detectors are designed to maximize the two detectors signal-to-noise (S/N) ratio and also reduce patient motion noise. The emitter/detector optics can be manufactured into the sensor body 31 by such methods as plastic injection molding technology. The projection/collection surfaces may be coated with a specular metallic film such as aluminum or composed of a high diffusely reflective material such as Dupont Teflon or Labsphere's Spectralon.

Electrical output signal from each of the sensor's two detectors are composed of two components. One component is a large non-pulsate DC signal due to light absorption of venous and arterial blood, skin, bone and surrounding tissue. The other component is a much smaller AC photoplethysmographic signal due to light absorption of the blood pulsate tissue. This signal output may be of the form of an analog current proportional to the input signal intensity using conventional silicon photo detectors. It may also be converted by a light to frequency (LTF) sensor manufactured by Texas Advanced Optoelectronic Solutions, Inc. (TAOS) to a square wave or pulse train whose frequency is linearly proportional to light intensity. These signals are sent by multimode fibers or electrical cable 44, 46 to the analyzer 50 input for further filtering and processing.

The analyzer 50 digitally processes the optical signals for removal of the DC signal component and further analog to digital (A/D) conversion applying standard techniques used in pulse oximetry by those skilled in the art. An example of this device may consist of a personal computer and Digital Signal Processor (DSP) board from Texas Instruments or standard oximeter electronics from such suppliers as Masimo or Nellcor. Conventional computational analysis may incorporate algorithms based on either an exactly determined or over-determined system of equations to calculate the total and ratio of concentrations of the hemoglobin derivatives and other blood analytes.

Output 52 may include display and audio-visual alarm electronics for “real time” results and digital storage using read-only memory (ROM) for digital storage (results, trends, alarms, etc.)

Digital/analog I/O 54 for monitor, chart reporting (transmitting data using WiFi, Bluetooth, network, direct printing, etc.) This information may be stored electronically in random access memory (RAM) or other digital storage media for retrieval at a later time.

1. Apparatus for the non-invasive, precision measurement of blood analytes concentration in pulsate, blood-perfused tissue comprising:
a source of a beam of interrogating electromagnetic radiation in the infrared region produceable in n selected bands, where n≧2, where each band interacts selectively with one of the blood analytes when said interrogating beam is incident upon the tissue to produce a diffusely reflected beam of said incident interrogating light,
a detector positioned between said source of electromagnetic radiation and the tissue, said detector being sensitive to radiation in said IR range, and said detector being curved concavely with respect to said tissue to maximize the detection over a large light collection area and produce an output electrical signal corresponding to the energy of said diffusely reflected beam, and
an analyzer that receives said signals and processes them to produce a measure of the concentration level of the blood analytes.

2. The apparatus of claim 1 wherein said light source is in the mid-infrared wavelength region, about 5 microns to about 10 microns.

3. The apparatus of claim 1 wherein said analyzer utilizes a residual least squares curve fitting algorithm to fit the collected diffuse light signals from the said blood pulsate tissue to a curve and an iterative constituent-sequenced algorithm for correlating said diffuse collected light signals with a set of the blood constituents.

4. The apparatus of claim 1 wherein said apparatus further includes a visual display of said measured concentration levels.

5. The apparatus of claim 1 wherein said apparatus further comprises the means for storing said measured current concentration levels.

6. Apparatus for the non-invasive, precision measurement of blood analytes concentration in pulsate, blood-perfused tissue comprising:
a source of a beam of interrogating electromagnetic radiation in the visible and near IR range produceable in n selected bands, where n≧2, where each band interacts selectively with one of the blood analytes where said interrogating beam is incident upon the tissue to produce both a diffusely reflected and a diffusely transmitted beam of said incident interrogating light,
a detector including a diffusely reflected light detector positioned on a first side of the tissue adjacent the light source, and a diffusely transmitted light detector positioned on the opposite side of the tissue from said light source, both said diffusely reflected and diffusely transmitted detectors being configured and positioned to collect, respectively, the diffusely reflected and diffusely transmitted light beams that have interacted with the tissue over a large collection area, each of said detectors producing an electrical signal corresponding to the energy of the diffused light so collected,
means for analyzing the output signals from said diffusely reflected and diffusely transmitted detectors; and
an analyzer that receives said signals and processes them to produce a measure of the concentration level of the blood analytes.

7. The apparatus of claim 6 wherein said detectors are curved concavely along a parabolic or ellipsoidal curvature that is concave with respect to the tissue.

8. The apparatus of claim 6 wherein said visible and near infrared light is in the range of approximately 400-1350 nm.

9. The apparatus of claim 8 wherein said visible and near infrared light is within the transparent tissue window from approximately 610 to 1311 nm.

10. The apparatus of claim 6 wherein said apparatus further includes a visual display of said measured concentration levels.

11. The apparatus according to claim 6 wherein said light source is selected from the group consisting of a quartz halogen lamp, a white light LED, discrete wavelength LED's, or diode lasers.

12. The apparatus according to claim 11, wherein said light source includes a spectrometer operating in combination with sources emitting a spectral continuum of light in the visible and near IR to produce said n spectral bands.

13. The method of non-invasively measuring with precision blood analytes concentrations comprising the steps of:
illuminating pulsate, blood-perfused tissue with an interrogating beam of infrared radiation in the mid-IR range and in n spectral bands where n≧2 and each band selectively interacts with a one of the analytes being measured,
detecting light diffusely reflected from said pulsate blood-perfused tissue wherein said detection is over a large collection area that is concavely curved with respect to the tissue and produces an output electrical signal corresponding to the intensity of the collected diffusely reflected light in each spectral band that in turn corresponds to the blood analyte concentration to be measured; and
analyzing said output signal of said detector to calculate the blood analyte concentration measurements.

14. The method according to claims 13 wherein said IR radiation region is in the mid-IR range and said analyzing utilizes a residual least squares curve fitting algorithm to fit the collected diffuse light signals from the said blood pulsate tissue to a curve and an iterative constituent-sequenced algorithm for correlating said diffuse collected light signals with a set of the blood constituents.

15. The method according to claim 13 wherein said analyzing collected light utilizes data over the full wavelength range of the interrogating IR beam.

16. The method of non-invasively measuring with precision blood analytes concentrations comprising the steps of:
illuminating pulsate, blood-perfused tissue with an interrogating beam of electromagnetic radiation in the visible and near IR range and in n spectral bands where n≧2 and each band selectively interacts with one of the analytes being measured,
detecting light that is both diffusely reflected and diffusely transmitted from said pulsate blood-perfused tissue, said detection beam is over a large collection area that is concavely curved with respect to the tissue, and producing an output electrical signal corresponding to the intensity of the collected diffusely reflected light in each spectral band and, in turn, corresponding to the blood analyte concentration to be measured; and
analyzing said output signal of said detector to calculate the blood analyte concentration measurements.

17. The method according to claim 16 wherein said range is approximately 400 to 1350 nm.

18. The method according to claim 16 wherein said range is within the transparent tissue window of approximately 610 to 1311 nm.

19. The method of claim 16 wherein said concavely curved detection is selected from the group consisting of ellipsoidal and parabolic.
